(19) United States
(12) Patent Application Publication
(10) Pub. No.: US 2002/0164285 A1
(43) Pub. Date: Nov. 7, 2002

(54) Kit containing tattoos for medical and surgical applications

(76) Inventors: Michelle S. Berryman, Marietta, GA (US); Deborah S. Wiese, El Cajon, CA (US)

Correspondence Address: Basam E. Nabulsi, CUMMINGS & LOCKWOOD, P.O. Box 1960, New Haven, CT, 06509-1960, US

(21) Appl. No.: 10/134,003

(22) Filed: Apr. 29, 2002

Related U.S. Application Data
(62) Division of application No. 09/788,953, filed on Feb. 20, 2001.

(51) Int. Cl. A61K 49/00; A61K 7/00

(52) U.S. Cl. 424/9.1; 424/401

(57) ABSTRACT

A method and system for applying temporary tattoos or other surface indications in locations remote from a region of medical/surgical treatment is disclosed. The method/system allows medical/surgical staff to indicate treatment and/or patient information in a non-invasive but reliable manner through temporary application of a tattoo or other surface indication at a location remote from a medical/surgical treatment region. The tattoos may include verbiage and/or schematic depictions, and advantageously convey information derived with respect to the patient/individual, e.g., allergies, physical conditions and the like. Utilization of the method/system of the present disclosure minimizes the likelihood of medical/surgical errors by effectively communicating information concerning individuals/patients to surgical/medical personnel.

### BACKGROUND

1. Technical Field

The present disclosure relates to a method and system for applying temporary tattoos or other surface indications in locations remote from a region of medical/surgical treatment, and more particularly to a method/system that allows medical/surgical staff to indicate treatment and/or patient information in a non-invasive but reliable manner through temporary application of a tattoo or other surface indication at a location remote from a medical/surgical treatment region.

2. Description of Background Art

An important aspect of medical/surgical treatment is accurate and reliable record keeping. For example, medical and surgical staffs devote considerable attention and energy to making appropriate notations on patient's charts and in patient's files/records. These notations are routinely reviewed in connection with ongoing treatment protocols and decisions, and provide critical background information as medical/surgical practitioners respond and diagnose current conditions/symptoms. The types of information routinely recorded in an individual's file/chart are numerous, including age, blood type, allergies, chronic conditions, and the types and locations of infections, tumors, strains, breaks, and the like.

A variety of medical/surgical personnel are routinely involved in administering care to patients. Thus, a medical/surgical office, laboratory or hospital may involve numerous clerks, attendants, nurses, nurse practitioners, physicians, surgeons and/or specialists in diagnosing a patient's condition and/or administering care to such patient. To some degree, these medical/surgical personnel consult with the patient to determine useful background information, e.g., nature of current symptoms, prior treatment regimens, etc. However, in large measure, medical/surgical personnel rely on the information contained in a patient's file or on a patient's chart to guide their treatment/diagnostic activities.

Despite the best efforts of medical/surgical personnel, circumstances have arisen where errors have been made in patient treatment/diagnosis through a failure to carefully review a patient's file and/or chart, and/or a failure to correctly recollect information contained in a patient's file and/or chart. Examples of such medical/surgical errors include operations/treatments performed on the incorrect portion of a patient's body, the administration of improper medicines or dosages, the transfusion of incorrect blood types, and a failure to properly take account of patient allergies, dietary restrictions, language barriers, religious convictions and/or preferences.

Efforts have been undertaken in the prior art to address the need to communicate medical-related information concerning an individual. Thus, for example, U.S. Pat. No. 4,156,539 to Davidson et al. discloses an identification card for use by athletes and sportsmen. The identification card includes information concerning the individual captured by perforated sections that are removable from the card. The perforated sections provide information concerning medical problems, such as diabetes, allergies and the like. A pressure adhesive is provided on the back of the identification card to facilitate attachment to an individual's clothing at the shoulder or other convenient location. Thus, the Davidson '539 identification card facilitates communication concerning an individual, e.g., with respect to a medical condition or a disability, if the individual is unable to communicate the information, e.g., due to accident, disability or the like.

A further prior art system is disclosed in U.S. Pat. No. 5,578,353 to Drew, III, and relates to a tattoo admission ticket. According to the Drew '353 system, a strip of general admission tickets is provided with a transferable “body tattoo” that may be used to identify that a person has paid admission. Each ticket in the disclosed strip includes a substrate and ink indicia coated thereon, the indicia being transferable to the skin of a ticket purchaser, e.g., by wetting with a transfer solution and pressing the ink indicia against the skin.

Uses of external markings have also been disclosed for certain medical/surgical procedures. Thus, U.S. Pat. No. 4,583,538 to Onik et al., U.S. Pat. No. 4,860,331 to Williams et al., and U.S. Pat. No. 5,306,271 to Zinreich et al. relate to placement of markings on a patient's body to facilitate biopsy. A stereotaxic biopsy placement system is disclosed the Onik '538 patent, an image marking device that may be adhered to the body for biopsy location is disclosed in the Williams '331 patent, and delineation of a biopsy region on the skin is disclosed in the Zinreich '271 patent. Similarly, U.S. Pat. No. 5,928,797 to Vineberg, which relates to a temporary tattoo device and method, refers to the potential use of the disclosed tattoo device to “allow[s] medical workers to temporarily mark an area of a patient's skin for a subsequent medical procedure.”

The prior art also includes numerous generic disclosures related to methods, systems and products for applying and/or creating visual images on the skin, e.g., through decals, tattoos that feature transfer sheets, controlled tanning techniques, adhesive stencils and non-reflective under eye applications. These general methods, systems and products are generally well known in the art.

Despite these many prior art disclosures and developments, a need remains for a method and/or system that facilitates communication of relevant information, e.g., information from the file or chart of the individual/patient, that might be of assistance to medical/surgical personnel. More particularly, a need remains for a method/system that communicates treatment information in a non-invasive, but reliable, manner. These and other needs are satisfied by the method and system of the present disclosure, as described hereinbelow.

### SUMMARY OF THE DISCLOSURE

The present disclosure provides a method and system for applying temporary tattoos or other surface indications in locations remote from a region of medical/surgical treatment, and more particularly to a method/system that allows medical/surgical staff to indicate treatment and/or patient information in a non-invasive but reliable manner through temporary application of a tattoo or other surface indication at a location remote from a medical/surgical treatment region. The method/system of the present disclosure further allows temporary application of indicia/information concerning an individual that is relevant to such individual, e.g., room location and the like.

According to the present disclosure, a method/system for conveying information concerning an individual is disclosed. The method/system preferably involves deriving information concerning a patient/individual, e.g., antibiotics and medicines to be avoided, body regions not to be included in the medical/surgical procedure and/or diagnostic/treatment regiment, and the like. The information may be derived from the chart or file of the patient/individual, by speaking with the patient/individual, or other appropriate means. Once the information is derived, a tattoo is advantageously selected that conveys the derived information. The tattoo may include verbiage, schematic depictions or combinations thereof The selected tattoo is then temporarily applied to the skin of the patient/individual in a body region remote from body region that is to be subject to medical attention. Thus, for example, the tattoo may be applied on the arm or leg that is not to be operated upon, the breast that is not to be biopsied, and the like.

Other features, aspects and advantages of the present disclosure are apparent from the detailed description which follows.

### BRIEF DESCRIPTION OF THE FIGURES

To assist those of ordinary skill in the relevant art to which the subject matter of the present disclosure relates to better understand the features, operations and uses hereof, reference is made to the attached figures and corresponding description, in which:

FIG. 1 is a schematic illustration of an exemplary temporary tattoo according to the present disclosure;

FIG. 2 is a schematic illustration of an exemplary alternative temporary tattoo according to the present disclosure;

FIG. 3 is a schematic illustration of an exemplary alternative temporary tattoo according to the present disclosure;

FIG. 4 is a schematic illustration of an exemplary alternative temporary tattoo according to the present disclosure;

FIG. 5 is a schematic illustration of an exemplary alternative temporary tattoo according to the present disclosure;

FIG. 6 is a schematic illustration of an exemplary alternative temporary tattoo according to the present disclosure;

FIG. 7 is a schematic illustration of an exemplary alternative temporary tattoo according to the present disclosure;

FIG. 8 is a schematic illustration of an exemplary alternative temporary tattoo according to the present disclosure; and

FIG. 9 is a schematic illustration of an exemplary alternative temporary tattoo according to the present disclosure.

### DETAILED DESCRIPTION OF PREFERRED EMBODIMENT(S)

According to the present disclosure, a method and system for applying temporary tattoos or other surface indications in locations remote from a region of medical/surgical treatment are provided. More particularly, a method/system that allows medical/surgical staff to indicate treatment and/or patient information in a non-invasive but reliable manner through temporary application of a tattoo or other surface indication at a location remote from a medical/surgical treatment region is disclosed.

For purposes of the detailed disclosure which follows, the term “tattoo” will be used to describe any temporary surface treatment or indication that may be applied to the skin of an individual, as are known in the art. Thus, the term “tattoo” encompasses traditional ink transfer mechanisms, e.g., temporary tattoo systems that utilize transfer sheets and the like, decals, and other image marking devices and systems, as are known in the art. Examples of temporary surface treatments of the types encompassed by the term “tattoo,” as used herein, are disclosed in U.S. Pat. Nos. 4,169,169; 4,175,151; 4,522,864; 4,594,276; 5,601,859; 5,676,401; 5,816,269; 5,958,560; and 6,074,721, the contents of which are hereby incorporated by reference.

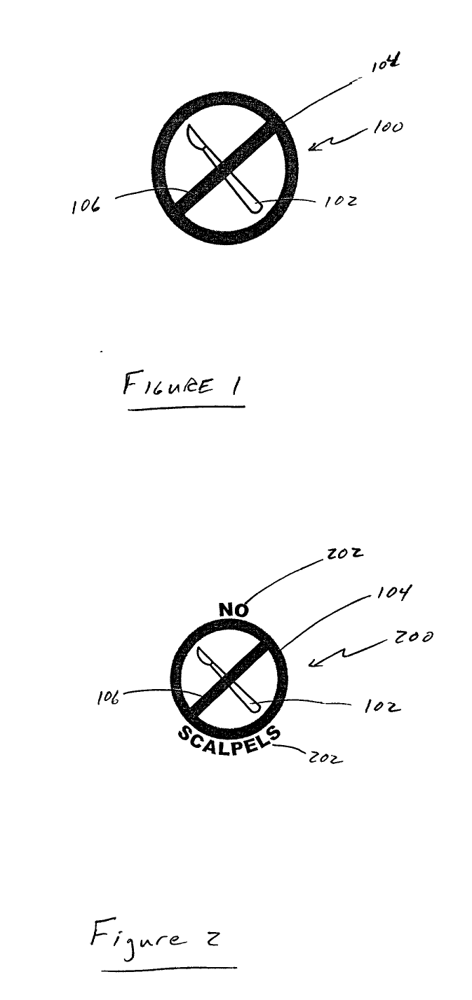

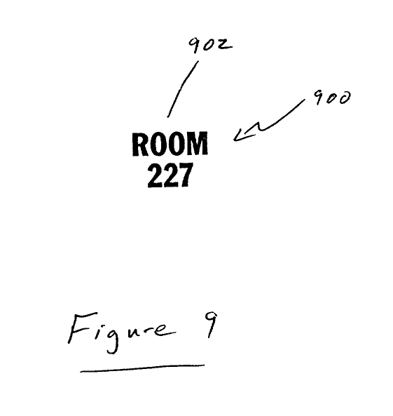

With reference to FIGS. 1-9, exemplary tattoos according to the present disclosure are illustrated. Thus, in FIG. 1, tattoo 100 includes a depiction or representation of a scalpel 102 enclosed within a circle 104 and a diagonal bisecting line 106. The combination of circle 104 and bisecting line 106 utilize the well-recognized convention for communicating a negative instruction, e.g., as is routinely employed on signs that communicate “no smoking”, “no eating”, “no skating”, and the like. According to the present disclosure, tattoo 100 is advantageously applied temporarily to the skin of an individual or patient to convey the fact that a scalpel is not to be utilized in the region of tattoo 100. It is contemplated that tattoo 100 applied by medical/surgical personnel or patient on body parts/extremities that are not to be the subject of operative procedure(s), e.g., the medically/surgically unintended arm, leg, chest or abdominal region of the individual/patient.

Tattoo 100 may be advantageously applied to the skin of a patient using known tattoo application techniques by medical/surgical personnel prior to surgery or in connection with providing further medical treatment to such patient, e.g., as part of a surgical preparatory procedure. Tattoo 100 preferably utilizes FDA-approved materials, e.g., FDA-approved inks and substrates, to enhance safety and efficacy. It is further preferred that the ink and substrate associated with tattoo 100 (and all disclosed tattoos herein) be fabricated using hypoallergenic materials, thereby minimizing the risk of infection or other adverse effect. The use of tattoo 100 may be guided by prior medical/surgical experiences in which an incorrect body region was improperly included in a medical/surgical procedure. Thus, a hospital or other health care facility may advantageously adopt standards of care to guide the utilization of tattoo 100, as will be readily apparent to persons skilled in the art.

With reference to FIG. 2, an alternative tattoo 200 is depicted that includes a depiction or representation of a scalpel 102 within a circle 104 and a bisecting line 106. However, unlike tattoo 100 depicted in FIG. 1, tattoo 200 includes ancillary verbiage 202. In the exemplary embodiment of FIG. 2, ancillary verbiage 202 communicates the message “NO SCALPELS” and is positioned above and below circle 102. However, as will be readily apparent to persons skilled in the art, ancillary verbiage 202 may take a variety of forms and communicate countless kernels of information and data. For example, ancillary verbiage 202 may be presented in multiple or alternative languages (e.g., Spanish, French, Chinese, etc.) or may convey information about the medical/surgical personnel involved in applying the tattoo (e.g., the practitioners name or identification number).

According to the present disclosure, tattoos 100, 200 are advantageously applied to the skin of an individual/patient in a region remote from the body region to be the subject of a medical/surgical procedure. It is contemplated that tattoos 100, 200 may never be viewed by the medical/surgical team involved in the actual medical/surgical procedure, only becoming visible if the medical/surgical team mistakenly turns its attention to the remote body region that is not to be involved in the medical/surgical procedure. Thus, utilization of tattoos 100, 200 allows medical/surgical personnel to communicate information concerning the patient in a reliable and non-invasive manner.

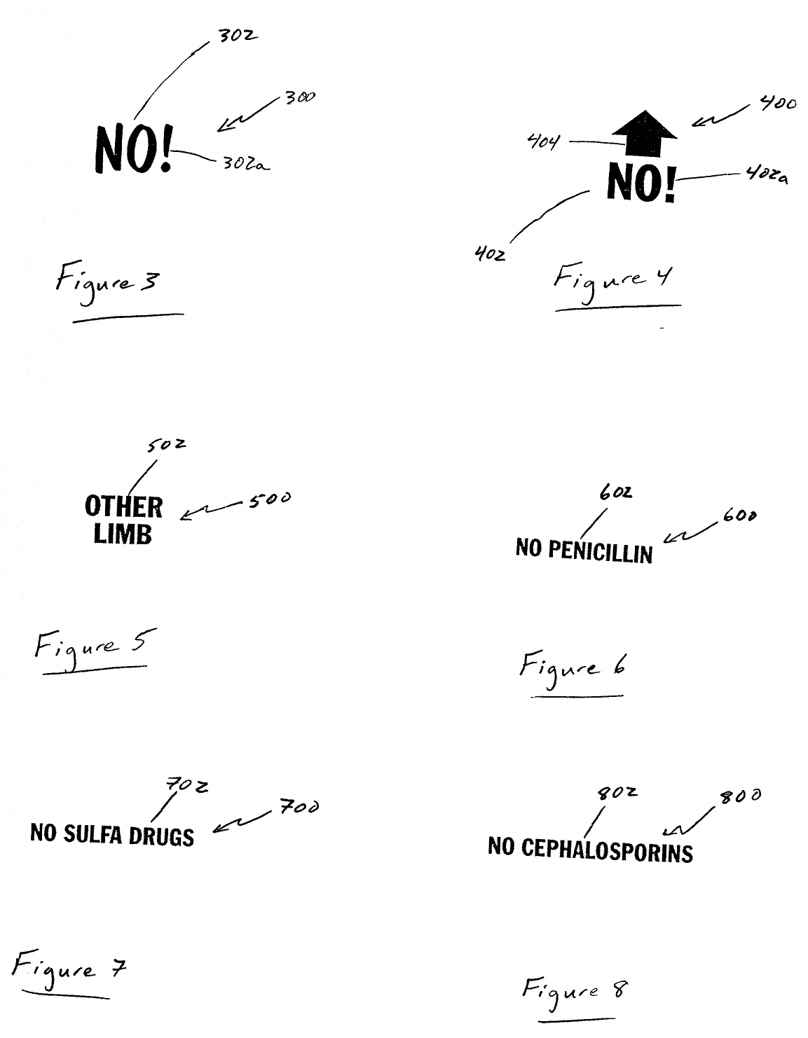

Turning to FIGS. 3 and 4, tattoos 300, 400 depict further alternative embodiments of the present disclosure. In each such embodiment, verbiage 302, 402 is depicted which, when applied to the skin of a patient/individual, provides useful information in the context of a medical/surgical procedure and/or diagnostic or treatment regimen. Verbiage 302, 402 is the unequivocal term “NO” and, in each depicted embodiment, includes punctuation 302a, 402a, namely an exclamation mark, which further clarifies the import of verbiage 302, 402. In the embodiment of FIG. 4, tattoo 400 includes location indicator 404, namely an arrow, which advantageously allows medical/surgical personnel to explicitly identify a body region/locale that is not to be addressed or included in a medical/surgical procedure or diagnostic/treatment regimen.

Tattoos 300, 400 may be used in a similar manner to tattoos 100, 200 described hereinabove. Thus, tattoos 300, 400 allow medical/surgical personnel to communicate useful information/data concerning a patient/individual, e.g., an arm not to be used for intravenous purposes, a leg/arm/body region not to be included in treatment, a breast not to be the subject of a biopsy, and the like. The generic nature of verbiage 302, 402 lends itself to widespread use, thereby minimizing the numbers/types of tattoos to be inventoried according to the present disclosure.

Turning to FIG. 5, tattoo 500 includes verbiage 502, namely “OTHER LIMB,” that is less generic than verbiage 302, 402 associated with tattoos 300, 400. Thus, tattoo 500 finds specific application according to the present disclosure in applications wherein medical/surgical personnel desire to communicate that the designated limb, i.e., leg or arm, is not to be the subject of a medical/surgical procedure and/or diagnostic or treatment regimen. As with prior tattoos disclosed herein, tattoo 500 is intended to be applied in a body region, i.e., on a limb, that is remote from the desired region of medical/surgical attention.

With reference to FIGS. 6-8, additional alternative tattoos 600, 700, 800 according to the present disclosure are depicted. Tattoos 600, 700, 800 include verbiage 602, 702, 802, respectively, that communicates information/data concerning a patient/individual, namely restrictions on medical treatments. In the case of tattoo 600, verbiage 602 states “NO PENICILLIN,” thereby conveying critical information concerning antibiotic restrictions associated with a particular patient/individual. While antibiotic restrictions of the type conveyed by verbiage 602 are routinely contained on the chart or within the file of a patient/individual, application of tattoo 600 in a region remote from the body region to be directly involved in the medical/surgical procedure lessens the likelihood that a treatment error may occur. Indeed, it is contemplated according to the present disclosure that standard(s) of care may be developed wherein tattoo(s) containing information concerning antibiotic/medicine restrictions will be located in a predetermined location, e.g., on the arm of the patient.

Tattoos 700, 800 include alternative verbiage 702, 802, namely “NO SULFA DRUGS” and “NO CEPHALOSPORINS,” respectively. Like the “NO PENICILLIN” verbiage included with tattoo 600, verbiage 702, 802 conveys important information concerning antibiotic/medicine restrictions associated with a particular patient/individual. Numerous alternative verbiages may be selected according to the present disclosure, e.g., “diabetic”, “anemic”, “HIV”, “A+”, “DNR”, “restricted diet”, “NKA”, etc. In each circumstance, valuable information concerning a patient/individual may be reliably and non-invasively communicated for ready access/consultation by medical/surgical personnel involved in patient/individual handling and/or treatment.

Turning to FIG. 9, a further alternative embodiment according to the present disclosure is depicted. Tattoo 900 includes verbiage 902, namely “ROOM 227.”According to the alternative embodiment of FIG. 9, it is contemplated that information/data concerning a patient/individual may be captured and communicated for ready access/consultation, particularly in circumstances where a patient/individual is suffering from conditions affecting memory function. Thus, for example, it is contemplated that patients/individuals suffering from Alzheimer's may have difficulty recalling the room in which they are housed, e.g., in a treatment center. Thus, tattoo 900 readily communicates such information, thereby avoiding confusion and enhancing efficiency of operations. Alternative information of the type conveyed by verbiage 902 is also contemplated according to the present disclosure, e.g., patient identification numbers, name, insurance coverage, etc.

It is further contemplated according to the present disclosure that a tattoo may be employed that provides a scannable code or inidicia, e.g., a bar code, that relates to a particular individual and/or condition. Thus, bar code tattoos may be temporarily applied to individuals, with each bar code corresponding to an individual and/or condition, e.g., diabetic, drug allergy, etc. In instances where individual bar codes correlate with specific individuals, e.g., a specific patient, a notation or entry of such bar code may be made on such individual's chart and/or in a computer database containing information concerning such individual. Once applied to the individual, the bar code could be easily scanned by medical/surgical personnel, e.g., nurses, clerks, attendants, and other medical practitioners, using handheld devices, e.g., a portable scanner associated with a PDA or other electronic instrument. In a preferred embodiment, scanning of an individual's bar code would provide access to relevant information concerning such individual, as described herein, and may allow entry into such individual's computerized record. Thus, temporary application of a tattoo that includes a bar code or other scannable indicia would allow medical/surgical staff to have ready access to relevant information concerning the individual in a reliable, efficient and cost effective manner.

Although the system/method of the present disclosure has been described with reference to several specific embodiments, the scope of the present disclosure is not to be restricted to the specifics of these exemplary embodiments. Thus, numerous alternative embodiments are contemplated and may be recognized that embody unique and advantageous aspects of the present disclosure. For example, it is contemplated that tattoos according to the present disclosure may be created in different fonts and font variations, different colors and/or in multi-colors, and that color coding systems may be developed for communicating information concerning patients/individuals. It is further contemplated that tattoos according to the present disclosure may be provided in kits, such kits containing a plurality of tattoos addressing different information/data concerning potential patients/individuals. Such alternative embodiments are to be included within the spirit and scope of the present disclosure.

1. A method for conveying information concerning an individual, comprising:
(a) deriving information concerning an individual;
(b) selecting a tattoo that conveys such derived information; and
(b) applying said tattoo to skin of said individual in a first body region remote from a second body region that is to be subject to medical attention.

2. A method for conveying information according to the method of claim 1, wherein said information concerning said individual is derived from a file or chart concerning said individual.

3. A method for conveying information according to the method of claim 1, wherein said information concerning said individual is derived from a discussion with said individual.

4. A method for conveying information according to the method of claim 1, wherein said tattoo is selected from a kit containing a plurality of tattoos.

5. A method for conveying information according to the method of claim 1, wherein said tattoo is selected from the group consisting of a temporary tattoo transferable from a transfer sheet and a decal.

6. A method for conveying information according to the method of claim 1, wherein said information concerns non-use of antibiotic or medicine with respect to said individual.

7. A method for conveying information according to the method of claim 1, wherein said information concerns non-treatment of said second body region.

8. A method for conveying information according to the method of claim 1, wherein said tattoo includes verbiage.

9. A method for conveying information according to the method of claim 1, wherein said tattoo includes a schematic depiction.

10. A method for conveying information according to the method of claim 9, wherein said schematic depiction includes a circle and diagonal bisecting line.

11. A method for conveying information according to the method of claim 1, wherein said information concerns medication dosage with respect to said individual.

12. A method for conveying information according to the method of claim 1, wherein said information concerns authorized treatment with respect to said individual.

13. A method for conveying information according to the method of claim 1, wherein said information is a scannable bar code.

14. A method for conveying information according to the method of claim 13, wherein said scannable bar code correlates to a computerized record concerning said individual.

15. A kit comprising:
(a) a container defining an enclosure of predefined dimensions;
(b) a plurality of tattoos positioned within said enclosure, each of said plurality of tattoos including predefined information and being adapted to be applied to skin of an individual in a first body region that is remote from a second body region that is to be subject to medical attention.

16. A method for conveying information concerning an individual, comprising:
(a) deriving residential information concerning an individual;
(b) selecting a tattoo that conveys such derived residential information; and
(c) applying said tattoo to skin of said individual.

17. A method for conveying information according to the method of claim 16, wherein said residential information includes a room number for said individual.
